(19) United States
(12) Patent Application Publication
(10) Pub. No.: US 2018/0370926 A1
(43) Pub. Date: Dec. 27, 2018

(54) PROCESS OF PREPARING TYROSINE KINASE INHIBITOR

(71) Applicant: PURDUE PHARMA L.P., Stamford, CT (US)

(72) Inventors: Jay Jie-Qiang WU, Fremont, CA (US); Ling WANG, Union City, CA (US)

(73) Assignee: PURDUE PHARMA L.P., Stamford, CT (US)

(21) Appl. No.: 15/750,588

(22) PCT Filed: Aug. 8, 2016
(86) PCT No.: PCT/US2016/046022

§ 371 Date: Feb. 6, 2018

Related U.S. Application Data
(60) Provisional application No. 62/202,610, filed on Aug. 7, 2015.

(51) Int. Cl. C07D 261/20

(52) U.S. Cl. CPC C07D 261/20; C07B 2200/13

(57) ABSTRACT

The invention relates to a process for preparing sodium 4-((3-(4-cyclohexylpiperazin-1-yl)-6-oxo-6H-anthra[1,9-cd]isoxazol-5-yl)amino)benzoate.

### FIELD OF THE INVENTION

The present disclosure relates to a process for preparing sodium 4-((3-(4-cyclohexylpiperazin-1-yl)-6-oxo-6H-anthra[1,9-cd]isoxazol-5-yl)amino)benzoate in ultra-high purity.

### SUMMARY OF THE INVENTION

The present disclosure presents a manufacturing process for preparing the following compound:

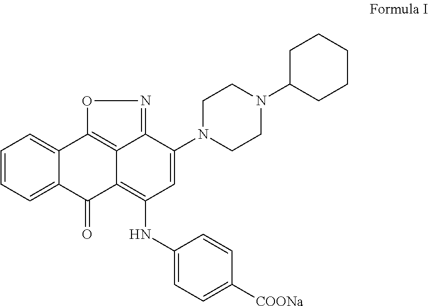

### Sodium 4-((3-(4-cyclohexylpiperazin-1-yl)-6-oxo-6H-anthra[1,9-cd]isoxazol-5-yl)amino)benzoate

Scheme 1 below provides the synthesis steps for making a compound of Formula II and the synthesis steps for making the compound of Formula I from the compound of Formula II.

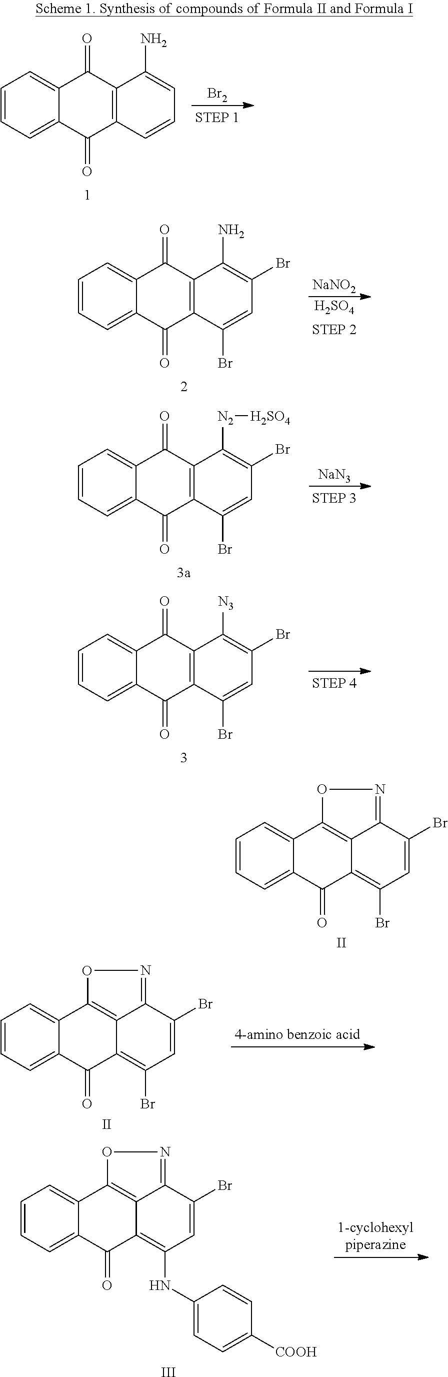

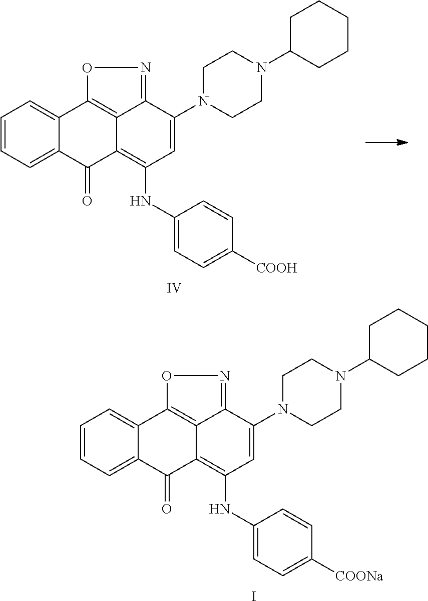

In one aspect, the present disclosure provides a process of preparing crystalline polymorph Form A of a compound of Formula I, sodium 4-((3-(4-cyclohexylpiperazin-1-yl)-6-oxo-6H-anthra[1,9-cd]isoxazol-5-yl)amino)benzoate:

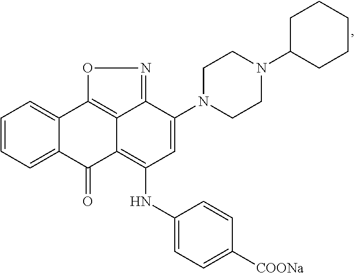

comprising:
(a) reacting 3,5-dibromo-6H-anthra[1,9-cd]isoxazol-6-one of Formula II:

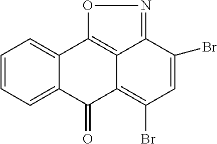 [SMILES: O=C1c2ccccc2-c2onc3c(Br)cc(Br)c1c23]

with 4-aminobeozoic acid to form 4-((3-bromo-6-oxo-6H-anthra[1,9-cd]isoxazol-5-yl)amino)benzoic acid of Formula III:

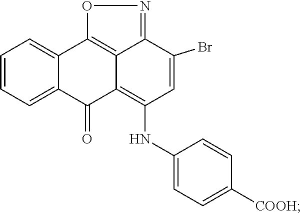 [SMILES: O=C(O)c1ccc(Nc2cc(Br)c3noc4c3c2C(=O)c2ccccc2-4)cc1]

(b) reacting 4-((3-bromo-6-oxo-6H-anthra[1,9-cd]isoxazol-5-yl)amino)benzoic acid (Formula III) and 1-cyclohexyl piperazine to form 4-((3-(4-cyclohexylpiperazin-1-yl)-6-oxo-6H-anthra[1,9-cd]isoxazol-5-yl)amino)benzoic acid of Formula IV:

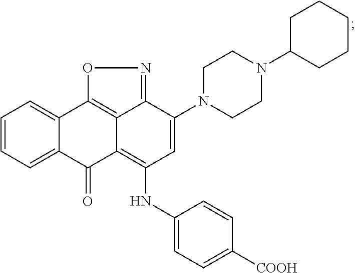 [SMILES: O=C(O)c1ccc(Nc2cc(N3CCN(C4CCCCC4)CC3)c3noc4c3c2C(=O)c2ccccc2-4)cc1]

and
(c) reacting 4-((3-(4-cyclohexylpiperazin-1-yl)-6-oxo-6H-anthra[1,9-cd]isoxazol-5-yl)amino)benzoic acid (Formula IV) and sodium hydroxide to form sodium 4-((3-(4-cyclohexylpiperazin-1-yl)-6-oxo-6H-anthra[1,9-cd]isoxazol-5-yl)amino)benzoate, the compound of Formula I.

In one aspect, the present disclosure provides a purification method of the compound of Formula I to improve the purity of a preparation of the compound of Formula I, for example, to over 99.9% (HPLC area %).

In one embodiment of this aspect of the invention, the compound of Formula IV obtained in step (b) is purified prior to using it in the next step (c). In another embodiment, the compound of Formula IV is purified by recrystallization. In another embodiment, the compound of Formula IV is purified by recrystallization from a solution comprising an acid and an organic solvent. In another embodiment, the acid is an inorganic acid, and preferably phosphoric acid or oxalic acid. In one embodiment, the acid is phosphoric acid. In another embodiment, the organic solvent is selected from the group consisting of N-methyl pyrrolidone, dimethylsulfoxide, methanol, and combinations thereof. In another embodiment, the solvent is N-methyl pyrrolidone.

In another embodiment, the recrystallization step of the compound of Formula IV further comprises adding an adsorption filtration medium to the solution. In one embodiment, the adsorption filtration medium is charcoal.

In one aspect, the present disclosure provides a method for purifying a compound of Formula IV, the method comprising recrystallizing the compound of Formula IV from a solution comprising an acid and an organic solvent. In one embodiment, the method further comprises adding an adsorption filtration medium to the solution.

In another aspect, the present disclosure provides a process for preparing polymorphic forms of the compound of Formula I.

In another aspect, the present disclosure provides a process for preparing the compound of Formula I in polymorphic Form A.

### BRIEF DESCRIPTION OF THE DRAWINGS

FIG. 1 is ¹H-NMR spectra of crystalline polymorph Form A of the compound of Formula I.

FIG. 2 is X-ray powder diffraction (XRPD) spectra of the crystalline polymorph Form A of the compound of Formula I.

FIG. 3 is Differential Scanning calorimetry (DSC) spectra of crystalline polymorph Form A of the compound of Formula I.

FIG. 4 is Raman spectra of crystalline polymorph Form A of the compound of Formula I.

FIG. 5 is IR spectra of crystalline polymorph Form A of the compound of Formula I.

### DETAILED DESCRIPTION OF THE INVENTION

The present disclosure provides a process for preparing the following compound of Formula I in a high purity, such as over 99.9% (HPLC are %):

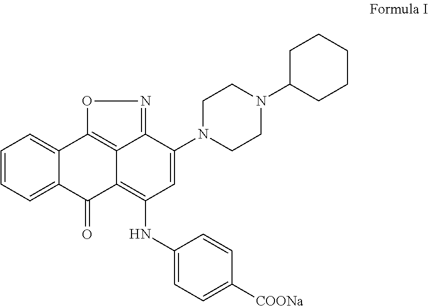

### Sodium 4-((3-(4-cyclohexylpiperazin-1-yl)-6-oxo-6H-anthra[1,9-cd]isoxazol-5-yl)amino)benzoate

In a specific embodiment, a process for the preparation of the compound of Formula I comprises the following steps:

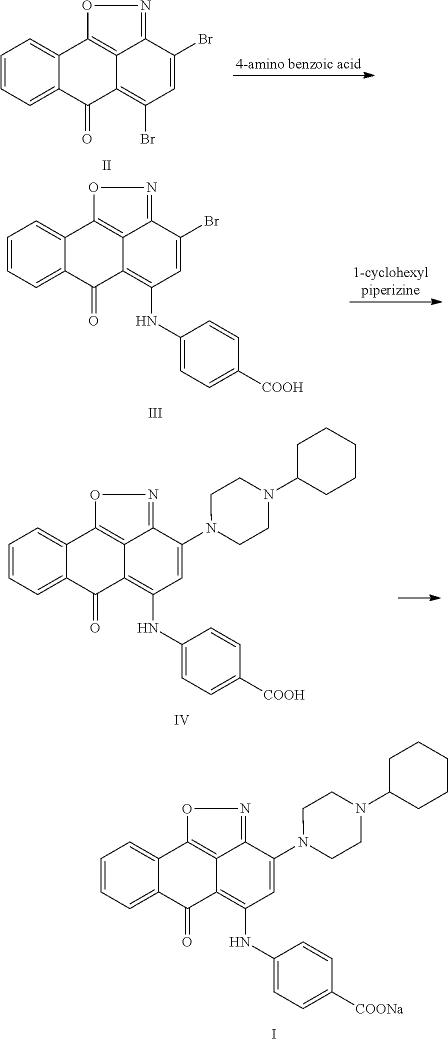

Accordingly, the present disclosure provides a process of preparing a compound of Formula I, sodium 4-((3-(4-cyclohexylpiperazin-1-yl)-6-oxo-6H-anthra[1,9-cd]isoxazol-5-yl)amino)benzoate:

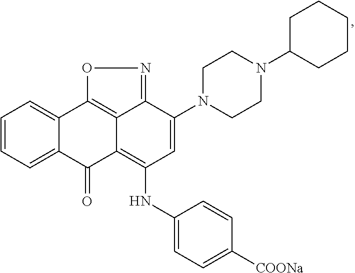

comprising:
(a) reacting 3,5-dibromo-6H-anthra[1,9-cd]isoxazol-6-one of Formula II:

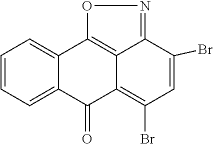 [SMILES: O=C1c2ccccc2-c2onc3c(Br)cc(Br)c1c23]

with 4-aminobenzoic acid to form 4-((3-bromo-6-oxo-6H-anthra[1,9-cd]isoxazol-5-yl)amino)benzoic acid of Formula III:

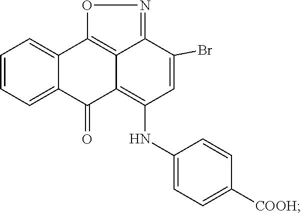 [SMILES: O=C(O)c1ccc(Nc2cc(Br)c3noc4c3c2C(=O)c2ccccc2-4)cc1]

(b) reacting 4-((3-bromo-6-oxo-6H-anthra[1,9-cd]isoxazol-5-yl)amino)benzoic acid (Formula III) and 1-cyclohexyl piperazine to form 4-((3-(4-cyclohexylpiperazin-1-yl)-6-oxo-6H-anthra[1,9-cd]isoxazol-5-yl)amino)benzoic acid of Formula IV:

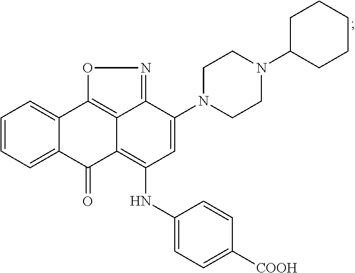 [SMILES: O=C(O)c1ccc(Nc2cc(N3CCN(C4CCCCC4)CC3)c3noc4c3c2C(=O)c2ccccc2-4)cc1]

and
(c) reacting 4-((3-(4-cyclohexylpiperazin-1-yl)-6-oxo-6H-anthra[1,9-cd]isoxazol-5-yl)amino)benzoic acid (Formula IV) and sodium hydroxide to form sodium 4-((3-(4-cyclohexylpiperazin-1-yl)-6-oxo-6H-anthra[1,9-cd]isoxazol-5-yl)amino)benzoate, the compound of Formula I.

In one embodiment, the reaction of the step (a) is performed at a temperature of from about 45° C. to about 70° C.; or at a temperature of from about 45° C. to about 55° C.

In another embodiment, the reaction of the step (b) is performed at a temperature of from about 50° C. to about 70° C.; or at a temperature of from about 60° C. to about 65° C.

In another embodiment, the reaction of the step (c) is performed at a temperature of from about 30° C. to about 60° C.; or at a temperature of from about 35° C. to about 45° C.; or at a temperature of from about 40° C. to about 45° C.; or at about 40° C.

In another embodiment, the compound of Formula III or Formula IV is prepared in a polar aprotic solvent in the presence of a base. In one embodiment, the polar aprotic solvent is selected from the group consisting of dimethyl acetamide and dimethyl sulfoxide. In one embodiment, the base is selected from the group consisting of lithium hydroxide and triethylamine.

In another embodiment, the process further comprising recrystallization of the compound of Formula IV from an acid and an organic solvent solution. In one embodiment, the acid is an inorganic acid. In another embodiment, the inorganic acid is phosphoric acid. In one embodiment, the organic solvent is N-methylpyrrolidone (NMP).

In another embodiment, the recrystallization step is performed at a temperature of from about 30° C. to about 70° C.; or from about 30° C. to about 50° C.

In another embodiment, the recrystallization step comprises the use of charcoal. In one embodiment, the charcoal is ECOSORB® C-941.

In another embodiment, the weight ratio of the solvent to the compound of Formula IV is from about 10:1 to about 20:1; or from about 12:1 to about 16:1.

In another embodiment, the weight ratio of phosphoric acid to the compound of Formula IV is from about 8:1 to about 20:1; or from about 10:1 to about 15:1.

In another embodiment, the compound of Formula I, as prepared, contains no more than about 0.1% total impurity.

In another embodiment, the compound of Formula I, as prepared, is a crystalline polymorph Form A.

It has been found that the process described above provides the compound of Formula I in a high purity when the intermediate compound of Formula IV is purified prior to using it in the step (c) of converting the free acid compound of Formula IV to its sodium salt of Formula I.

In one embodiment, the compound of Formula IV is purified by recrystallization before using it in the step (c). In another embodiment, the compound of Formula IV is purified by recrystallization from a solution comprising an acid and an organic solvent.

In one aspect, the present disclosure provides a process of preparing a compound of Formula I:

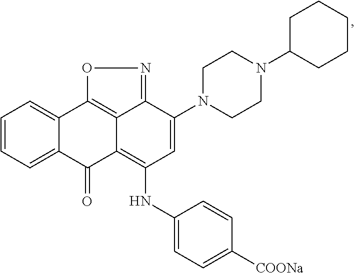

comprising:
(a) reacting a compound of Formula II:

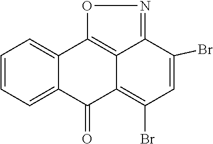 [SMILES: O=C1c2ccccc2-c2onc3c(Br)cc(Br)c1c23]

with 4-amino benzoic acid to form a compound of Formula III:

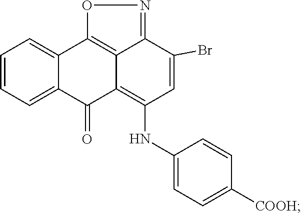 [SMILES: O=C(O)c1ccc(Nc2cc(Br)c3noc4c3c2C(=O)c2ccccc2-4)cc1]

(b) reacting the compound of Formula III and 1-cyclohexyl piperazine to form a compound of Formula IV:

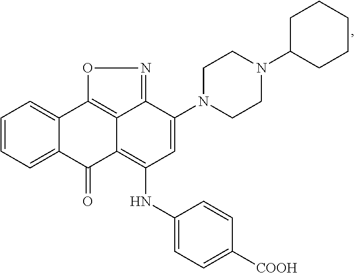 [SMILES: O=C(O)c1ccc(Nc2cc(N3CCN(C4CCCCC4)CC3)c3noc4c3c2C(=O)c2ccccc2-4)cc1]

and
   purifying the compound of Formula IV by recrystallization; and
(c) reacting the compound of Formula IV obtained in step (b) and sodium hydroxide to form the compound of Formula I.

In one embodiment, the compound of Formula IV is isolated after the reaction in the step (b) and purified by recrystallization from a solution comprising an acid and an organic solvent. Typically, the isolated compound of Formula IV is dissolved in the solution of an acid and an organic solvent at a temperature under about 40° C. In another embodiment, the acid used in the recrystallization step is an inorganic acid. In another embodiment, the inorganic acid is phosphoric acid or oxalic acid. In another embodiment, the acid is phosphoric acid. In one embodiment, the weight ratio of the acid to the compound of Formula IV in the recrystallization is from about 1:8 to about 1:20. In another embodiment, the weight ratio of the acid to the compound of Formula IV is from about 1:10 to about 1:15. In another embodiment, the weight ratio of the phosphoric acid to the compound of Formula IV is from about 1:8 to about 1:20. In another embodiment, the weight ratio of the phosphoric acid to the compound of Formula IV is from about 1:10 to about 1:15.

In one embodiment, the organic solvent used in the recrystallization step is selected from the group consisting of N-methyl pyrrolidone, dimethylsulfoxide, methanol, and combinations thereof. In another embodiment, the solvent is N-methyl pyrrolidone. In one embodiment, the weight ratio of the organic solvent, and preferably N-methyl pyrrolidone, to the compound of Formula IV in the recrystallization is from about 10:1 to about 20:1. In another embodiment, the weight ratio of the organic solvent, and preferably N-methyl pyrrolidone, to the compound of Formula IV is from about 12:1 to about 16:1. The compound of Formula IV can be crystallized from the solution by adding water.

Adsorption filtration media can be used in the recrystallization step to absorb impurities. In one embodiment, the recrystallization of the compound of Formula IV further comprises treating the solution with an adsorption filtration medium. In another embodiment, the adsorption filtration medium is charcoal, such as ECOSORB® C-941. Typically, the adsorption filtration medium is added to the solution in the recrystallization step, preferably in admixture with the organic solvent, and the mixture is stirred, for example 2-3 hours, after which the adsorption filtration medium is removed by filtering. The compound of Formula IV can be crystallized from the filtrate by adding water.

In one aspect, the present disclosure provides a method for purifying a compound of Formula IV, the method comprising recrystallizing the compound of Formula IV from a solution comprising an acid and an organic solvent as described above. In one embodiment, the method further comprises treating the solution with an adsorption filtration medium, such as charcoal, prior to crystallizing the compound of Formula IV.

In one embodiment, intermediate compounds of Formula III and IV are prepared according to a process of the present invention in a polar aprotic solvent in the presence of a base. Polar aprotic solvents suitable for the present methods include the polar aprotic solvents well known in the art, and the examples include, but are not limit to, dimethyl acetamide (DMAc), dimethyl formamide (DMF), dimethyl sulfoxide (DMSO), acetonitrile (ACN), and the like. In one embodiment, the solvents in use of the present invention are selected from dimethyl acetamide and dimethyl sulfoxide. The bases suitable for the present method can be hydroxide or alkyl amine, such as lithium hydroxide and triethyl amine (TEA), respectively.

In a specific embodiment, the preparation process of the compound of Formula III comprises:
I. Mixing 4-amino benzoic acid and a base in a polar aprotic solvent at a temperature of about 40° C. In one embodiment, the base is lithium hydroxide. In one embodiment, the solvent is DMAc.
II. Portion-wisely adding 3,5-dibromo-6H-anthra[1,9-cd]isoxazol-6-one into the solution. Then raising the temperature to about 45° C. to 70° C., or about 45° C. to 55° C.
III. Maintaining the reaction mixture for about 18 to 20 hours.
IV. Slowly adding methyl t-butyl ether (MTBE) to the reaction mixture and then cooling to about 0° C. to 10° C., or about 2° C. to 6° C.
V. Isolating the solid and drying the product at room temperature to get 4-((3-bromo-6-oxo-6H-anthra[1,9-cd]isoxazol-5-yl)amino)benzoic acid.

In another embodiment, the preparation process of the compound of Formula IV comprises:
I. Dissolving 4-((3-bromo-6-oxo-6H-anthra[1,9-cd]isoxazol-5-yl)amino)benzoic acid in a polar aprotic solvent. In one embodiment, the solvent is DMSO.
II. Then adding a base and 1-cyclohexyl piperazine into the solution. In one embodiment, the base is (TEA).
III. Raising the temperature to about 50° C. to 70° C., or about 60° C. to 65° C.
IV. Maintaining the reaction mixture for about 2 to 4 hours.
V. Then slowly adding methyl t-butyl ether and methanol solution to the reaction mixture, and cooling to ambient temperature.
VI. Isolating and washing the wet cake with methyl t-butyl ether and methanol (MeOH) solution, then drying.
VII. Dissolving the crude product in an acid and organic solvent solution under about 40° C.

The organic solvents include, but are not limit to, N-methyl pyrrolidone (NMP), DMSO, methanol, and the like. In one embodiment, the solvent is NMP. The acid is selected from phosphoric acid (PPA), oxalic acid and the like. In one embodiment, the acid is PPA.
VIII. Adding adsorption filtration media, for example, charcoal, to absorb impurities, wherein adsorption filtration media or charcoal is well known in the art. In one embodiment, the charcoal is ECOSORB® C-941.
IX. Then adding water and filtering the solid to get 4-((3-(4-cyclohexylpiperazin-1-yl)-6-oxo-6H-anthra[1,9-cd]isoxazol-5-yl)amino)benzoic acid.

In another embodiment, the preparation process of the compound of Formula I comprises:
I. Mixing 4-((3-(4-cyclohexylpiperazin-1-yl)-6-oxo-6H-anthra[1,9-cd]isoxazol-5-yl)amino)benzoic acid in sodium hydroxide and methanol solution.
II. Raising the temperature to about 30° C. to 60° C., or about 40° C.
III. Maintaining the reaction for about 1-4 hours.
IV. Lowering the temperature to ambient temperature and filtering the solid to get the product, sodium 4-((3-(4-cyclohexylpiperazin-1-yl)-6-oxo-6H-anthra[1,9-cd]isoxazol-5-yl)amino)benzoate.

In another aspect of the present invention, the compound of Formula II can be prepared by the following process:

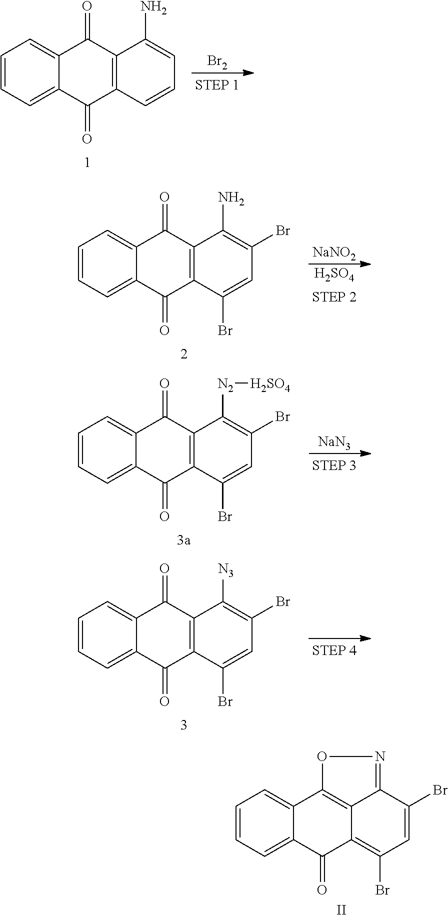

In a specific embodiment, the preparation process of the compound 2 comprises:
a) Mixing 4-amino-10-hydroxyanthracen-9(10H)-one in a polar organic solvent, for example, methanol.
b) Adding bromine slowly to the suspension at about 50° C. to 60° C. for about 1 hour.
c) Maintaining the reaction mixture at 50° C. to 60° C. for 18 to 24 hours.
d) Cooling the reaction mixture to around 10° C. to 20° C.
e) Filtering and washing the wet cake with MTBE (methyl tert-butyl ether). The solid was dried under ambient temperature to give compound 2.

In a specific embodiment, the preparation process of the compound 3a comprises:
a) Cooling concentrated H₂SO₄ to about 0° C.
b) To the cooled concentrated H₂SO₄, adding NaNO₂ slowly at the rate of maintaining a gentle evolution of brown gas.
c) After the addition of NaNO₂, maintaining the mixture at about 35° C. for 1 hour.
d) Adding compound 2 to the mixture portionwise. And then stirring resulting mixture at about 50° C. to 55° C. for about 3 to 6 hours.
e) Cooling reaction mixture to ambient temperature, and then pouring it into crushed ice.
f) Collecting solid under vacuum filtration, washing wet cake with ice-water, followed by mixture of ethanol/MTBE (1/1). The product can be used for the next step without further purification.

In a specific embodiment, the preparation process of the compound 3 comprises:
a) Adding compound 3a to the solution of NaN₃ in water slowly at room temperature.
b) Stirring the mixture at ambient temperature for about 16 to 24 hours.
c) Slowly adding 6N NaOH aqueous solution.
d) Collecting the solid by vacuum filtration. Washing the wet cake with water multiple times.
e) Re-suspending the filtrate cake in water for about 15 to 30 minutes. Then filtering under reduced pressure, and washing with water, and followed by the mixture of acetone/water (9/1), air dry to give compound 3.

In a specific embodiment, the preparation process of the compound of Formula II comprises:
a) Adding compound 3 to toluene at about 70° C. portionwise over 1 hour.
b) Stirring the mixture at about 70° C. for about 16 to 24 hours.
c) Cooling the reaction temperature to ambient temperature.
d) Collecting the solid product by vacuum filtration. Washing the filter cake with MeOH.
e) Suspending the solid in MeOH at ambient temperature for about 1 hour. Then filtering to obtain the compound of Formula II.

The use of the terms “a”, “an”, “the”, and similar referents in the context of this disclosure (especially in the context of the claims) are to be construed to cover both the singular and the plural, unless otherwise indicated. Recitation of ranges of values herein are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure and is not a limitation on the scope of the disclosure unless otherwise claimed. No language in the specification should be construed as indicating any non-claimed element as essential to the practice of the disclosure.

The term “about,” as used herein, includes the recited number±10%. Thus, “about 10” means 9 to 11.

The terms “adsorption filtration media” and “adsorption filtration medium” are used herein interchangeably to refer to a composition containing one or more adsorbents, such as, for example, activated charcoal (such as ECOSORB C-941), calcium silicate, magnesium silicate, activated alumina, zeolites, and ion exchange resins.

The terms “activated charcoal” and “charcoal” are used herein interchangeably.

The present invention is further illustrated in, but not limit to, the following examples.

### Example 1

### Preparation of 4-((3-bromo-6-oxo-6H-anthra[1,9-cd]isoxazol-5-yl)amino)benzoic acid

About 19 kg of 4-amino benzoic acid and about 5.5 kg of lithium hydroxide were mixed in about 200 L of DMAc. About 25 kg of 3,5-dibromo-6H-anthra[1,9-cd]isoxazol-6-one was then slowly mixed in the pre-mixed DMAc solution. The reaction was under Nitrogen protection at about 45-55° C. for about 18-20 hours. After reaction completion was confirmed by HPLC, about 7.5 L of MTBE was slowly added to the reactor. Reaction mixture was then slowly cooled to about 2-6° C. under nitrogen protection. Centrifuged the solid and washed with about 4.5 L of MTBE. Dried the wet cake under about 25-30° C. to obtain about 17.3 kg of 4-((3-bromo-6-oxo-6H-anthra[1,9-cd]isoxazol-5-yl)amino)benzoic acid.

### Example 2

### Preparation of 4-((3-(4-cyclohexylpiperazin-1-yl)-6-oxo-6H-anthra[1,9-cd]isoxazol-5-yl)amino)benzoic acid

About 17.3 kg of 4-((3-bromo-6-oxo-6H-anthra[1,9-cd]isoxazol-5-yl)amino)benzoic acid was mixed with about 238 L of DMSO. About 11 L of TEA and 12 kg of 1-cyclohexyl piperazine were added to the reaction. Temperature was then raised to about 60-65° C. After 2-3 hours, slowly added about 116 L of MTBE and MeOH (10:1) solution and adjusted the temperature to 40-50° C. The solid was centrifuged and washed by 22.5 L of MTBE and MeOH (10:1) solution and followed by about 22 L of MeOH. Solid was dried under reduced pressure at about 25-30° C. for 12-24 hours.

About 1.8 kg phosphoric acid was dissolved in 90 L of N-methyl pyrrolidone (NMP). Previously obtained crude product was dissolved in about 163 L of NMP. Under about 40° C., two solutions were mixed together for 1-2 hours. Then about 5.5 kg of ECOSORB® C-941 in about 20 L of NMP was added to the previously mixed solution. The mixture was stirred for another 2-3 hours under nitrogen protection before filtration. About 600 L of purified water was slowly added to the solution under about 40° C. Solid was centrifuged and washed with about 21 L of water and followed by about 56 L of MTBE. Solid was dried under reduced pressure at about 25-30° C. for 8-12 hours to obtain the 4-((3-(4-cyclohexylpiperazin-1-yl)-6-oxo-6H-anthra[1,9-cd]isoxazol-5-yl)amino)benzoic acid at about 98% purity and about 91% yield.

### Example 3

### Preparation of sodium 4-((3-(4-cyclohexylpiperazin-1-yl)-6-oxo-6H-anthra[1,9-cd]isoxazol-5-yl)amino)benzoate

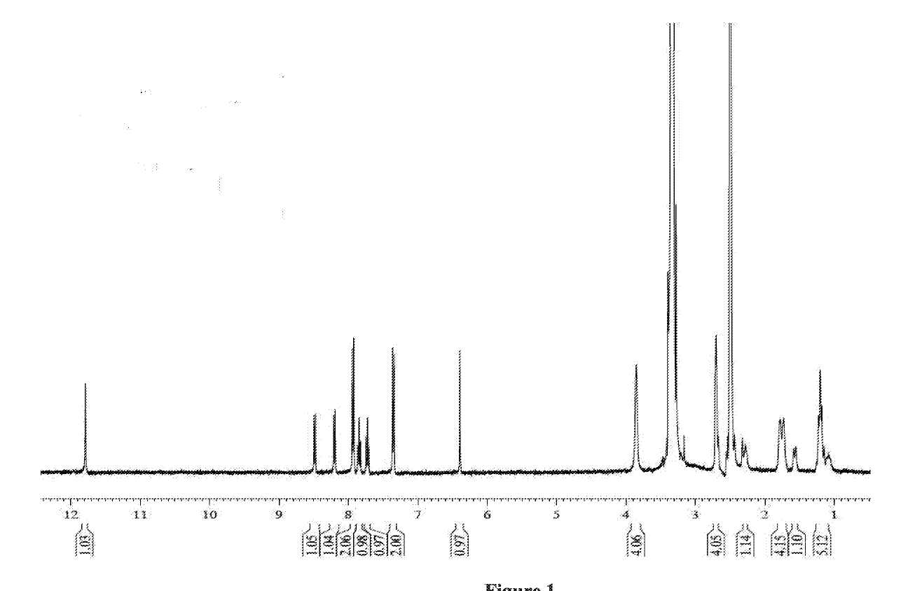

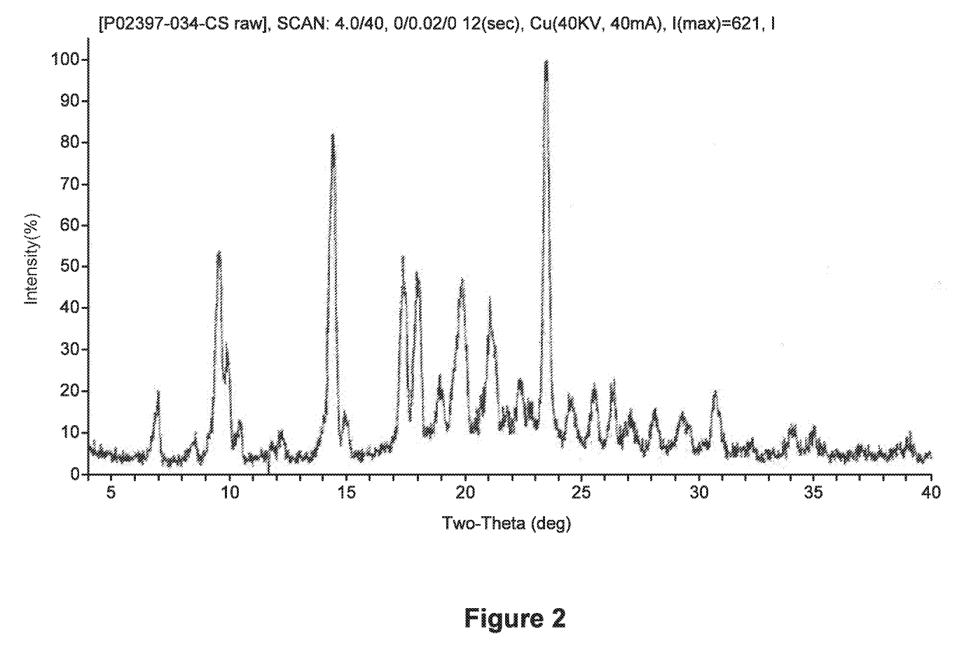

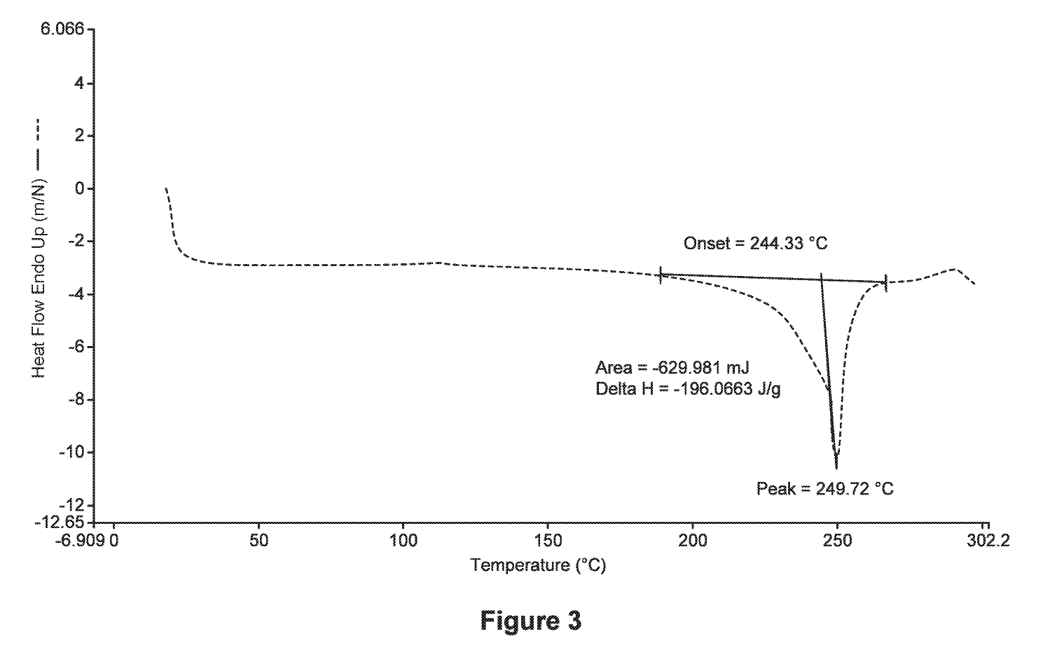

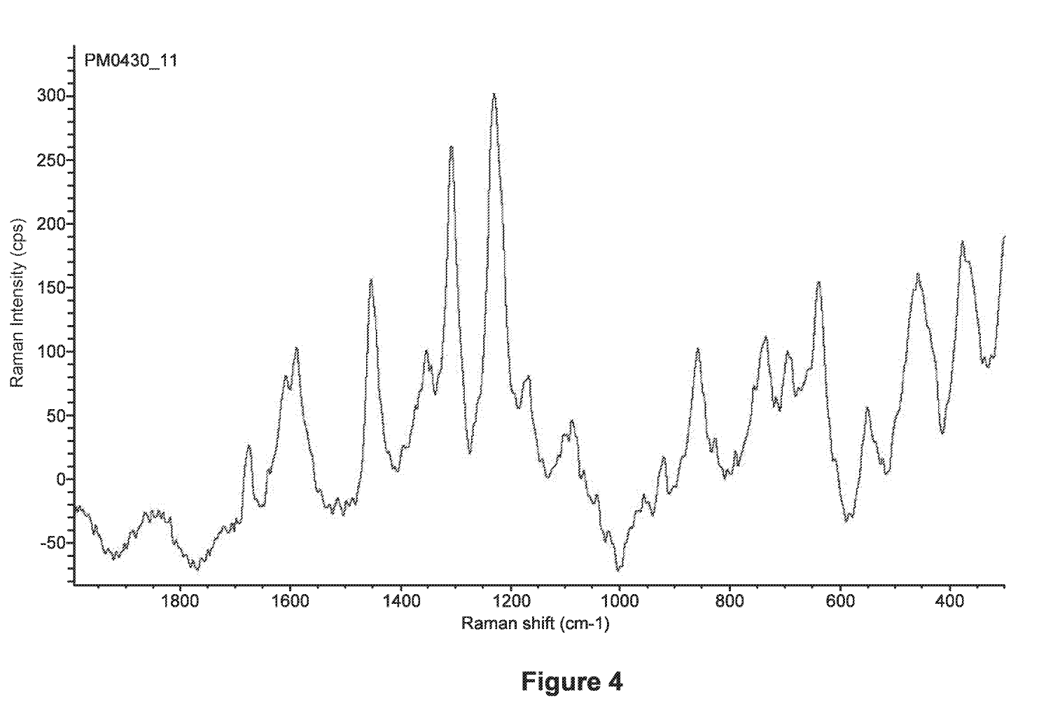

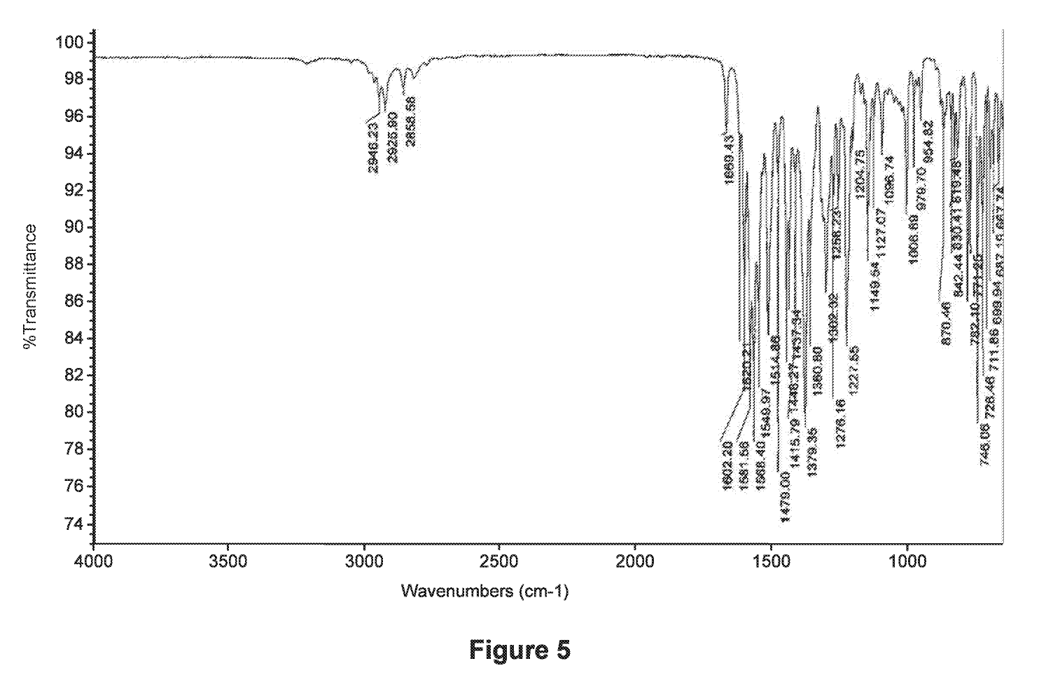

About 21 kg of 4-((3-(4-cyclohexylpiperazin-1-yl)-6-oxo-6H-anthra[1,9-cd]isoxazol-5-yl)amino)benzoic acid was slurred in 625 L of 0.4 M NaOH in MeOH and purified water (4:1) solution under about 40-45° C. for about 2-4 hours. Then slowly cooled the reaction to room temperature and stirred for another 2-4 hours. After confirmed the reaction completion by HPLC, the solid was centrifuged and washed with about 57 L of MTBE. The wet cake was re-suspended in about 245 L of 0.1M NaOH in MeOH/H₂O solution under room temperature. The wet cake was centrifuged and washed with about 56 L of MTBE again. The filtered solid was re-suspended in about 250 L of MTBE under room temperature for about 1-2 hours. The solid was separated and dried at 25-30° C. under reduced pressure for 12-24 hours to obtain the final product with purity more than 99.9% (HPLC) and about 90% yield. Mass spectra give [M+1]=523.2. ¹H-NMR (400 MHz, DMSO-d6, see FIG. 1), ppm (δ): 11.79 (1H, s), 8.48 (1H, d), 8.20 (1H, d), 7.93 (2H, d), 7.84 (1H, t), 7.72 (1H, t), 7.35 (2H, d), 6.39 (1H, s), 3.85 (4H, m), 2.72-2.70 (4H, m), 2.28-2.265 (1H, m), 1.72-1.78 (4H, m), 1.55-1.58 (1H, m), 1.08-1.23 (5H, m). Sodium content: 3.8%. The Raman spectrum is shown in FIG. 4, and the XRPD data is given in Table 1 below and FIG. 2, IR spectra is given in FIG. 5 and DSC spectra is given in FIG. 3. These data confirm the stable crystalline polymorphic form (Form A) of the compound of Formula I.

TABLE 1
XRPD Table of the polymorphic form,
Form A of the compound of Formula I.

| | Angle | d-value | Intensity |
|---|---|---|---|
| # | 2-Theta° | (Angstrom) | (%) |
| 1 | 7.160 | 12.3354 | 14.3 |
| 2 | 8.757 | 10.0899 | 22.4 |
| 3 | 9.820 | 9.0001 | 70.1 |
| 4 | 10.161 | 8.6986 | 88.1 |
| 5 | 10.522 | 8.4006 | 7.3 |
| 6 | 12.459 | 7.0985 | 14.5 |
| 7 | 14.641 | 6.0454 | 82.0 |
| 8 | 15.219 | 5.8170 | 22.8 |
| 9 | 17.680 | 5.0124 | 41.0 |
| 10 | 18.240 | 4.8598 | 73.0 |
| 11 | 19.104 | 4.6420 | 14.2 |
| 12 | 20.220 | 4.3883 | 100.0 |
| 13 | 21.381 | 4.1525 | 33.3 |
| 14 | 22.579 | 3.9347 | 10.1 |
| 15 | 23.721 | 3.7478 | 99.7 |
| 16 | 24.898 | 3.6733 | 11.8 |
| 17 | 25.761 | 3.4555 | 17.8 |
| 18 | 25.522 | 3.3581 | 13.6 |
| 19 | 27.161 | 3.2804 | 17.7 |
| 20 | 28.321 | 3.1487 | 10.5 |
| 21 | 29.481 | 3.0273 | 12.8 |
| 22 | 29.781 | 2.9976 | 8.7 |
| 23 | 30.478 | 2.9306 | 7.7 |
| 24 | 30.921 | 2.8896 | 18.6 |
| 25 | 34.281 | 2.6137 | 12.1 |
| 26 | 35.120 | 2.5532 | 8.0 |
| 27 | 35.483 | 2.5279 | 6.0 |

### Example 4

### Preparation of Compound 2

30.0 kg of 4-Amino-10-hydroxyanthracen-9(10H)-one was suspended in MeOH (70 L). To the suspension, 53.7 kg of bromine was added slowly to the suspension at 60° C. about 1 hr. After the addition of bromine, the reaction mixture was vigorously stirred at 50-60° C. for about 18-24 hours. The reaction mixture was then cooled to around 18° C. The resulting suspension was filtered, washed with 210 L of MTBE. The red solid was dried in air to give product 2 as a red solid (˜50.0 kg, yield 98%). HPLC analysis showed 96% purity. ¹H-NMR (CDCl₃, 300 Hz) δ 8.26 (m, 2H), 8.09 (s, 1H), 7.80 (m, 2H).

### Example 5

### Preparation of Compound 3a

27.8 L of concentrated H₂SO₄ was cooled to 0° C. Then 2.99 kg of NaNO₂ as solid was added to concentrate H₂SO₄ portionwise at the rate of maintaining a gentle evolution of brown gas. After the addition of NaNO₂, the mixture was stirred at 35° C. for 1 hr. Compound 2 (15.0 kg) was then added to the mixture portionwise. The resulting mixture was stirred at 50-55° C. for 4 h. After cooled to room temperature, the reaction mixture was poured into crushed ice (25 kg). The yellow solid was precipitated out. The solid was collected by vacuum filtration, washed with 5˜10 L of ice-water, followed by 10 L of 1:1 mixture of ethanol/MTBE to give a wet solid, which was dried in air. 24.7 kg of the crude damp product was obtained. The product was used in the next step without further purification

### Example 6

### Preparation of Compound 3

To a 100 L jacketed reactor was charged the solution of NaN₃ (2.73 kg) in water (50 L). Compound 3a was added portionwise at room temperature (caution: vigorous gas evolution was observed when compound 3a was added and subsided slowly in 5-10 minutes). After the addition, the mixture was stirred at room temperature overnight. An aqueous solution of NaOH (6N, 3.0 L) was then added slowly to the mixture. Then the solid was collected by vacuum filtration and washed with water (2×3.0 L). The filtrate cake was slurred with water (10 L) for about 15 minutes, filtered, and washed with water (5 L), and followed by the mixture of acetone/water (9/1, 18.0 L), air dried to give crude compound 3 (23.2 kg of damp solid). HPLC analysis showed 95% purity. ¹H-NMR (300 MHz, DMSO-d₆) δ 8.51 (s, 1H), 8.11-8.15 (m, 2H), 7.91-7.94 (m, 2H).

### Example 7

### Preparation of the Compound of Formula II

26.5 kg of crude compound 3 was added portionwise over 1 h to 50 L of toluene at 70° C. The mixture was then stirred at 70° C. overnight. After the reaction temperature was cooled to RT, the solid product was collected by vacuum filtration. The filter cake was washed with 6 L of MeOH. The solid obtained was re-suspended in 5 L of MeOH and stirred at room temperature for about 1 hour. After filtration, 13.3 kg of the wet product of compound of Formula II was obtained as a yellow solid. HPLC analysis showed 98.7% purity with the largest single impurity of 0.7%. ¹H-NMR (300 MHz, DMSO-d₆): δ 8.27 (m, 2H), 8.13 (m, 1H), 7.91 (m, 1H), 7.78 (m, 1H).

X-Ray Powder Diffraction (XRPD).

Approximately 2 mg of sample was gently compressed on the XRPD zero back ground single obliquely cut silica sample holder. The sample was then loaded into a D/MAX 2200 X-ray powder diffractometer (Rigaku) or a Philips X-Pert MPD diffractometer and analyzed using the following experimental conditions (Tube anode: Cu; Generator tension: 40 kV; Tube current: 40 mA; Wavelength alpha1: 1.54056 Å; Wavelength alpha2: 1.5444 Å; Start angle [2 theta]: 5; End angle [2 theta]: 50; and Continuous scan). For suspected novel forms a slightly slower scan speed was used over a range of 4-40°2ϑ.

Raman Spectroscopy.

Samples were analyzed by a Nicolet Almega XR Dispersive Raman Microscope for its Raman spectrum using the following conditions (Exposure Time: 1.0 s; Acquisition No: 10; Pinhole Size: 25, 50 or 100 μm; Wavelength range: 2000˜300 cm⁻¹ (single grating); Laser: He—Ne 780 nm 100% power; Objective: 20×/0.40 or 50×/0.75 (magnifier/numerical aperture number)). Then the measured Raman spectra were corrected by baseline subtraction using the software OMNIC™ v7.3.

Simultaneous Thermal Analysis (STA).

Approximately 5 mg of sample was accurately weighed into a ceramic crucible and it was placed into the chamber of Perkin-Elmer STA 600 TGA/DTA analyzer at ambient temperature. The sample was then heated at a rate of 10° C./min from 25° C. to 300° C. during which time the change in weight was monitored as well as DTA signal. The purge gas used was nitrogen at a flow rate of 20 cm³/min.

Differential Scanning Calorimetry (DSC).

Approximately, 5 mg of each sample was weighed into an aluminum DSC pan and sealed non-hermetically with an aluminum lid. The sample was then loaded into a Perkin-Elmer Jade DSC and held at 25° C. Once a stable heat-flow response was obtained, the sample was then heated to 300° C. at a scan rate of 10° C./min and the resulting heat flow response was monitored. A 20 cm³/min helium purge was used. Prior to analysis, the instrument was temperature and heat flow verified using an indium standard.

Polarised Light Microscopy (PLM).

An Olympus BX50 microscope, equipped with an analyser and polariser, was used to observe each sample under polarised light. Micrographs of the sample were taken by using a JVC-TKC1380 digital camera connected to a PC running Studio QuickStart version 9.3.2. A 20×/0.5 (magnifier/numerical aperture (NA) value) objective was used to view samples and capture images.

Gravimetric Vapor Sorption (GVS).

Approximately 20 mg of sample was placed into a wire-mesh vapor sorption balance pan and loaded into an ‘IgaSorp’ vapor sorption balance (Hiden Analytical Instruments). The sample was then dried by maintaining a 0% humidity environment until no further weight change was recorded. Subsequently, the sample was then subjected to a ramping profile from 0-90% RH at 10% RH increments, maintaining the sample at each step until equilibration had been attained (99% step completion). Upon reaching equilibration, the % RH within the apparatus was ramped to the next step and the equilibration procedure repeated. After completion of the sorption cycle, the sample was then dried using the same procedure. The weight change during the sorption/desorption cycles were then monitored, allowing for the hygroscopic nature of the sample to be determined.

All publications and patent applications herein are incorporated by reference to the same extent as if each individual publication or patent application was specifically and individually indicated to be incorporated by reference. The foregoing detailed description has been given for clearness of understanding only and no unnecessary limitations should be understood therefrom as modifications will be obvious to those skilled in the art. It is not an admission that any of the information provided herein is prior art or relevant to the presently claimed inventions, or that any publication specifically or implicitly referenced is prior art.

Embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A process of preparing a compound of Formula I: 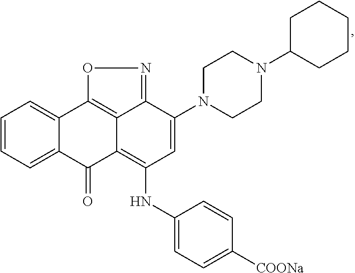
comprising:
(a) reacting a compound of Formula II: 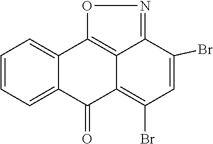 [SMILES: O=C1c2ccccc2-c2onc3c(Br)cc(Br)c1c23]
with 4-aminobenzoic acid to form a compound of Formula III: 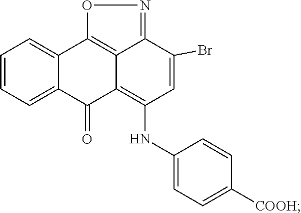 [SMILES: O=C(O)c1ccc(Nc2cc(Br)c3noc4c3c2C(=O)c2ccccc2-4)cc1]
(b) reacting the compound of Formula III and 1-cyclohexyl piperazine to form a compound of Formula IV: 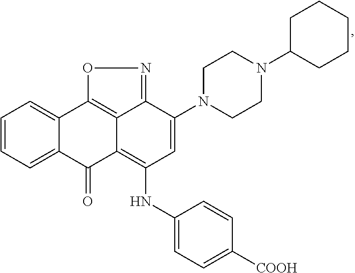 [SMILES: O=C(O)c1ccc(Nc2cc(N3CCN(C4CCCCC4)CC3)c3noc4c3c2C(=O)c2ccccc2-4)cc1]
and
purifying the compound of Formula IV by recrystallization; and
(c) reacting the compound of Formula IV and sodium hydroxide to form the compound of Formula I.

2. The process of claim 1, wherein the compound of Formula IV is purified by recrystallization from a solution comprising an acid and an organic solvent.

3. The process of claim 2, wherein the acid is an inorganic acid.

4. The process of claim 2 or claim 3, wherein the inorganic acid is phosphoric acid or oxalic acid.

5. The process of any one of claims 2 to 4, wherein the inorganic acid is phosphoric acid.

6. The process of any one of claims 2 to 5, wherein the weight ratio of the acid to the compound of Formula IV is from about 1:8 to about 1:20.

7. The process of any one of claims 2 to 6, wherein the weight ratio of the acid to the compound of Formula IV is from about 1:10 to about 1:15.

8. The process of any of claims 2 to 7, wherein the organic solvent is selected from the group consisting of N-methyl pyrrolidone, dimethylsulfoxide, methanol, and a combination thereof.

9. The process of any one of claims 2 to 8, wherein the organic solvent is N-methyl pyrrolidone.

10. The process of any one of claims 2 to 9, wherein the weight ratio of the organic solvent to the compound of Formula IV is from about 10:1 to about 20:1.

11. The process of any one of claims 2 to 10, wherein the weight ratio of the organic solvent to the compound of Formula IV is from about 12:1 to about 16:1.

12. The process of any one of claims 2 to 11, further comprising adding water to crystallize the compound of Formula IV.

13. The process of any one of claims 2 to 12, comprising treating the solution with an adsorption filtration medium prior to crystallizing the compound of Formula IV.

14. The process of claim 13, wherein the adsorption filtration medium is charcoal.

15. The process of claim 14, wherein the charcoal is ECOSORB® C-941.

16. The process of any one of claims 1 to 15, wherein the reaction of step (a) is performed at a temperature of from about 45° C. to about 70° C.; or at a temperature of from about 45° C. to about 55° C.

17. The process of any one of claims 1 to 16, wherein the reaction of step (b) is performed at a temperature of from about 50° C. to about 70° C.; or at a temperature of from about 60° C. to about 65° C.

18. The process of any of claims 1 to 17, wherein the reaction of step (c) is performed at a temperature of from about 30° C. to about 60° C.; or at a temperature of from about 35° C. to about 45° C.; or at a temperature of from about 40° C. to about 45° C.; or at about 40° C.

19. The process of any of claims 1 to 18, wherein the compound of Formula III or Formula IV is prepared in a polar aprotic solvent in the presence of a base.

20. The process of claim 19, wherein the polar aprotic solvent is selected from the group consisting of dimethyl acetamide and dimethyl sulfoxide.

21. The process of claim 19 or 20, wherein the base is selected from the group consisting of lithium hydroxide and triethylamine.

22. The process of any of claims 1 to 21, wherein the recrystallization is performed at a temperature of from about 30° C. to about 70° C.; or from about 30° C. to about 50° C.

23. The process of any one of claims 1 to 22, wherein the recrystallization is performed at a temperature under about 40° C.

24. The process of any of claims 1 to 23, wherein the compound of Formula I contains no more than about 0.1% total impurity.

25. The process of any of claims 1 to 24, wherein the compound of Formula I is crystalline polymorph Form A.
